(19) United States
(12) Patent Application Publication
(10) Pub. No.: US 2014/0308373 A1
(43) Pub. Date: Oct. 16, 2014

(54) Herbal Extract for Inhibiting Growth of Tumor

(71) Applicants: Wen-Guang Fong, (US); Ming-Che Ting, Taoyuan City (TW); Cing-Huo Liao, Fenglin Township (TW)

(72) Inventor: Wen-Guang Fong, Fenglin Township (TW)

(73) Assignees: Wen-Guang Fong, Fenglin Township (TW); Ming-Che Ting, Taoyuan City (TW); Cing-Huo Liao, Fenglin Township (TW)

(21) Appl. No.: 14/192,001

(22) Filed: Feb. 27, 2014

(30) Foreign Application Priority Data
Apr. 11, 2013 (TW) 102112921

(51) Int. Cl. A61K 36/47; A61K 36/185

(52) U.S. Cl. CPC A61K 36/47; A61K 36/185
USPC 424/731

(57) ABSTRACT

A herbal extract for inhibiting the growth of tumor described herein and the herbal extract is obtained by extracting a mixture of white castor and Formosanum elderberry with water and/or alcohol under a heating condition, in which a weight ratio of white castor is 1 to 99% and a weight ratio of Formosanum elderberry is 99 to 1%. The herbal extract is useful in preventing, treating, relieving, and improving the quality-of-life of patients suffering from cancer.

### FIELD OF THE INVENTION

The present invention is generally related to a herbal extract, and more particularly to a herbal extract for inhibiting growth of tumor.

### BACKGROUND OF THE INVENTION

There is one new cancer patient in Taiwan for every 5 minutes and 48 seconds in 2010, according to the Department of Health (DOH) of Taiwan. The DOH also reports that there are 90,649 new cancer patients in total in 2010 to reach the highest all-time record, and the disease of cancer has been the top cause of death in Taiwan for the past 31 years.

The disease of cancer is occurred because of uncontrollably growing of human's cells that are not to be dead but become immortalized. Normal cells in the body follow an orderly path of growth, division, and death. A programmed death of cell is called apoptosis, and when this process breaks down, cancer begins to form. Accordingly, the immortalized cells growing beyond their normal limit will invade adjacent tissues and the malignant cells may also metastasize to spread to other locations in the body via the bloodstream or lymphatic system. Therefore, cancer cells often form a mass tissue known as a tumor.

There are many different causes of cancer including; carcinogens, age, genetic mutations, immune system problems, diet, weight, lifestyle, andenvironmental factors such as pollutants. Some viruses such as a human papilloma virus (HPV) that is implicated in cervical cancer and some bacterial infections are also known to cause cancers.

There are many well-know cancer treating methods that are being implemented today. Some of them include the standard methods of cancer treatments such as surgery, chemotherapy and radiation therapy. The choice of therapy depends upon the location and grade of the tumor, the situation of the disease, and the general condition of a person's health as well. Surgery is used for removing the visible tumor and is effective when the cancer is small and confined. However, the metastasis situation that cancers already invade the adjacent tissue or spread to distant sites often limits effectiveness of surgery. Chemotherapy, using drugs to kill cancer cells, can be used for destroying cancer cells that are hard to detect and that have been spreading. The effectiveness of chemotherapy is often limited by toxicity to tissues of the body. Radiation, designed to kill cancer cells, can be applied externally or internally of the body, but also can cause damage to normal tissue.

There are other alternative methods available for cancer treatment that are applied by integrating traditional treating manners to help the cancer patient, and sometimes to be recommended for alleviating some side effects of treatments caused in chemotherapy and radiation. In these alternative treatments, it includes a treatment by use of traditional medical herb. In theory and practice, the traditional medical herb is completely different from the modern western medical treatment in that the traditional medical herb is applied by considering the way that the tissues of human body work with each other, the way that the human body falls illness, and that processes to treat the illness. The treating theory of the traditional medical herb is for treating a whole human body rather than a specific portion of the human body. The traditional medical herb is used for treating (I) the vitality of human body by tonic herbs, (II) the cancer by anti-cancer herbs and (III) the side effects caused by radiation therapy and chemotherapy.

Therefore, the formula of the traditional medical herb usually comprises components extracting from raw herbs with a very small quantity. The theory pursues the goal of achieving synergism among these different components. One advantage of this kind of synergism theory is that it avoids the chance of excessive toxicity that is happened when any one in these components is given in large quantity. This is in sharp contrast to the modern western medical treatment where a pharmacologically active molecule is given in a large dose, which is a maximally tolerated dose, to target one physiologic endpoint.

### SUMMARY OF THE INVENTION

It becomes an important issue regarding how to provide a herb for inhibiting a growth of tumor and a herbal extracts that can be taken by a user conveniently.

A castor plant (Ricinus communis) belongs to a Euphorbia Family (Euphorbiaceae), where there are diverse species as a kind of economically-important flowering plants. It is the only member of the genus Ricinus, and it has no immediate relatives. It is a robust annual that may grow 2 to 5 meters in one season with full sunlight, heat and adequate moisture. In areas with mild, frost-free winters, it may live for many years and become quite woody and tree-like. The large, palmately lobed leaves may be over 50 cm. The stalked leaves usually consist of eight radiating, pointed leaflets with slightly serrated edges and prominent central veins. Flowers occur most of the year in dense terminal clusters (inflorescences), with female flowers just above the male flowers. This species is clearly monoecious, with separate male and female flowers on the same individual. There are no petals and each female flower consists of a little spiny ovary (which develops into the fruit or seed capsule), and a bright red structure with feathery branches (stigma lobes) that receives pollen from male flowers. Each male flower consists of a cluster of many stamens which literally smoke as they shed pollen in a gust of wind. The spiny seed pod or capsule is composed of three sections or carpels which split apart at maturity. Each section (carpel) contains a single seed. When the carpel dries and splits open, the seed is often ejected with considerable force. The seeds (and to a much lesser extent the leaves) contain ricin, a protein, which is highly toxic in small quantities.

Ricin can be targeted to specific unwanted cells, such as cancer cells, by conjugating the Ricin toxin A-chain (RTA subunit) to antibodies or growth factors that preferentially bind the unwanted cells. These immunotoxins have worked very well for vitro applications, e.g. bone marrow transplants.

In bone marrow transplant procedures, RTA-immunotoxins have been used successfully to destroy T lymphocytes in bone marrow taken from histocompatible donors. This reduces rejection of the donor bone marrow, a problem called “graft-vs-host disease” (GVHD). With regard to steroid-resistance, in an acute GVDH situation, it is found RTA-immunotoxins can help to alleviate the condition. Also, for a patient in autologous bone marrow transplantation, the patient's own bone marrow is treated anti-T cell immunotoxins to destroy malignant T-cells existing in T cell leukemias and lymphomas.

Formosanum Elderberry (Sambucus formosanum Nakai), belonging to Caprifoliaceae, has been used in Taiwan as a folk remedy for men's health although it smells really bad. Another name of Formosanum Elderberry is said as Sambucus Chinensis. Their roots and leaves can be used for remedying twist, sprain and strain, and reduce swelling and asepticize. They also can be used for dissolving phlegm, dispelling stasis, relaxing muscles and joints and stimulating blood circulation as well. By the way, the leaves can treat gonorrhea. With leaves covered on the bodies for external treatment, they can sterilize wound, reduce swelling, and waist sprain. The roots, stems, and leaves have the function of removing heat, detoxifying, and treating carbuncle-abscess and swelling.

Furthermore, Formosanum Elderberry can endure blaze and semi-shade. It can grow in subtropical regions and warm temperate zones. It prefers growing and sowing in moist soil. Its flowering phase is from late spring to summer (May. to Sep.) and its florescence is from May to July. The fruits are bacciform, spherical, and gamboge when they are ripe. The mature period of fruits is from September to December.

Therefore, an object of the present invention is to provide a herbal extract for inhibiting a growth of tumor. The herbal extracts comprise a combination of herbal extracts derived from plants of Formosanum Elderberry and white castor. The herbal extracts provided herein can be administered to an individual patient who suffers from cancerous conditions to improve a situation of symptoms associated with these diseases.

According to the present invention, a herbal extract for inhibiting tumor growth is obtained by extracting a mixture of white castor and Formosanum elderberry by use of water and/or alcohol under a heating condition, in which a weight ratio of white castor is 1 to 99% and a weight ratio of Formosanum elderberry is 99 to 1%.

According to the present invention, in the heating condition of extracting the mixture of white castor and Formosanum elderberry by use of water, the weight ratio of white castor to Formosanum elderberry is within the range of 1:1 to 1:2.

According to the present invention, in the heating condition of extracting the mixture of white castor and Formosanum elderberry by use of water, the weight ratio of white castor to Formosanum elderberry is within the range of 2:1 to 1:1.

According to the present invention, the heating condition of extracting the mixture of white castor and Formosanum elderberry by use of water is processed at a temperature above 70° C.

According to the present invention, the heating condition of extracting the mixture of white castor and Formosanum elderberry by use of water is processed at a temperature above 70° C. for over 60 minutes.

According to the present invention, a cancer is the one selected from a group consisting of leukemia, lymphoma, Hodgkin lymphomas, non-Hodgkin lymphomas, colorectal cancer, pancreatic cancer, breast cancer, ovarian cancer, prostate cancer, thyroid cancer, renal cell carcinoma, hepatoma, bile duct carcinoma, esophageal cancer, nasopharyngeal carcinoma, lung carcinoma, and any combination thereof.

According to the present invention, the herbal extract is used for reducing a size of a tumor.

According to the present invention, the herbal extract is in a form of a oral dosage.

According to the present invention, the oral dosage is in a form of a liquid, a powder, a tablet, a capsule or a multiparticulate dosage form.

According to the present invention, the herbal extract is obtained by extracting a mixture of white castor, Formosanum elderberry and pig bone by use of water and/or alcohol under a heating condition, in which a weight ratio of white castor is 1 to 99%, a weight ratio of Formosanum elderberry is 99 to 1%, and a weight ratio of pig bone is 99 to 1%.

The extracts of the present invention possess the effect of reducing the size of the tumor, inhibiting tumor metastasis, inhibiting to some extent of tumor growth, abating to some extent for one or more symptoms associated with cancer, stabilizing the growth of the tumor, extending the time to disease progression, improving overall survival, and increasing the quality of life.

### DETAILED DESCRIPTION OF THE PREFERRED EMBODIMENTS

The foregoing and other aspects of the present invention will now be described in more detail with respect to the description and methodologies provided herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing embodiments only but is not intended to limit the invention.

In one embodiment the herbal composition is an aqueous solution of herbal extracts. In another embodiment the herbal composition is a mixture of the herbal extracts in powdered form. The extracts may be aqueous or organic solvent extracts of the herbs. The extracts may be in liquid or powdered form.

An extract refers to the residue of soluble solids obtained after an herb, or selected part thereof is for example chopped, crushed, pulverized, minced, or otherwise treated to expose its maximum surface area and to be placed in intimate contact with a liquid, usually but not necessarily, under conditions of agitation and elevated temperature. Then, after a period of time under these conditions, the mixture is filtered to remove solids to obtain the liquid material. In other embodiment, the liquid obtained is further processed by, for example but not limitation, evaporation or freeze drying. The liquid for the extracting process may be water or an organic solvent, for example but not for limitation, an alcohol.

### Embodiment 1

White castor and Formosanum elderberry are picked and washed with cold water, and then the water is drained off from the two herbs, in which the white castor, preferably the root of the white castor, and the Formosanum elderberry are mixed in a weight ratio 1:1 before are put in a pot. In a preferable embodiment, a weight of the white castor and the Formosanum elderberry is 210 gram respectively. And then add an amount of pig bone in a way such as 30 gram into the pot. Add about five bowls of water, which is approximately 1200 mL to thus the water level is high enough to cover all of the herbs materials. Stewing them at above 70° C. over at least 60 minutes by a small fire to evaporate out most of the water. The concentrated herbal extract provided herein can be administered to individuals.

### Example 1

Chang, female patients, complained her loss of appetite for over three months. Her chest X-ray showed mediastinal nodules for examination in the hospital. The mediastinal nodules was diagnosed as diffuse large cell lymphoma by pathological examination. She accepted CHOP (cyclophosphamide, doxorubicin, vincristine, and prednisolone) chemotherapy in the Division of Hematology-Oncology of the hospital. After Chang received six times chemotherapy, she had symptoms including a poor appetite, abdominal distension, dry skin, and hair loss. The assessment of positron emission tomography scanning indicated that there was still residual tumors existing. According to the available blood test data, Chang before chemotherapy was RBC:5.1×10⁶/μl, WBC:5450/μl, PLT:223×10³/μl, Hb:15.5 g/dl, Hct:43.9%, MCH:30.4 pg. The data of Chang in chemotherapy final stage was WBC:995/μl, PLT:53×10³/μl, Hb:13.3 g/dl. Chang discharged from hospital then she took the herbal extracts as described in the first embodiment. She then went back to the hospital for a blood test a few weeks later, and her data is showed as WBC:7040μl, PLT:570×10³/μl, Hb:13.5 g/dl. Before the first beginning of the treatment of chemotherapy, the patient had a normal amount of blood cells (WBC:5450/μl). By the process of chemotherapy that the patient received a variety of combinative chemotherapy drugs in the hospital, therefore she showed side effects of bone marrow suppression, while the number of her white blood cells (WBC:995/μl) was downed in the chemotherapy final stage. However, after she left the hospital and started to administer the herbal composition of the first embodiment, the data of WBC as 1230μl in the beginning is increased 7040/μl. Her health is recovered fast, and her tumor is changed to be smaller. This shows the herbal extract in the first embodiment of the present invention has a significant role in preventing bone marrows suppression.

### Embodiment 2

White castor and Formosanum elderberry are washed with cold water and then the water is drained from the two herbs. The white castor and the Formosanum elderberry are mixed in a weight ratio 1:2 into a pot. And then add an amount of pig bone into the pot. Add about five bowls of water to cover all over the herbs and materials. Stewing them with at above 70° C. over at least 60 minutes by a small fire until most of the water is evaporated out. The concentrated herbal extract provided herein can be administered to individuals.

### Example 2

Patient Chao is a 63-year-old man who firstly developed vague abdominal discomfort. When the pain persisted, his physician referred him for a CT scan, which revealed enlarged lymph nodes in neck, groin, abdominal and aorta. The pathology report described the enlarged lymph nodes was consistent with non-Hodgkin's lymphoma. Chao then completed four cycles of chemotherapy, but the chemotherapy revealed poor treatment efficacy for him. Then Chao discharged from hospital, and he took the herbal extracts in the second embodiment of the present invention. A few weeks later, his fever is stopped. He then goes back to the hospital by lymphatic Photography (Lymphangiogram), and his right cervical and inguinal lymph nodes were clear. His spirits become sparkled and his physical situation is improved to even allow him for outdoors activities. Before he received chemotherapy, his white blood cell count of 5900/μl was normal, but the count values were reduced to degree of 950/μl during a course of treatment. However, after he took herbal extracts in the second embodiment of the present invention continuously after he left the hospital, his white blood cell count is raised to 6540/μl. That indicates the herbal extract in the second embodiment of the present invention has a significant role in preventing bone marrows suppression.

### Embodiment 3

White castor and Formosanum elderberry are washed with cold water and then the water is drained from the two herbs. The white castor and the Formosanum elderberry are mixed in a weight ratio 2:1 into a pot. And then add an amount of pig bone into the pot. Add about five bowls of water to cover all over the herbs and materials. Stewing them at above 70° C. over at least 60 minutes by a small fire to evaporate most of the water out. The concentrated herbal extract provided herein can be administered to individuals.

### Example 3

Patient Chan is a 54-year-old man with a syndrome of recurrent cold and persistent fever. One day, he found there was a lump just above his left clavicle bone suddenly. He went for a biopsy and it was clearly indicated Hodgkin lymphomas. Then Chen went to the hospital through one more surgery to remove multiple lymph nodes near the spinal cord and the spleen. Furthermore, he accepted an entire course of chemotherapy following the surgery, and he started to experience significant chemotherapy-related side effects. Chan tried the medical herbal extracts of the third embodiment following the end of chemotherapy. The herbal extract improves his symptoms of a dry mouth (xerostomia), upset stomach, constipation, diarrhoea and other side effects of chemotherapy. He claims feeling good and stronger and more powerful than ever. No abnormal abdominal lymph node enlargements of his abdomen is presently checked by ultrasonography and positron emission tomography. Before he received chemotherapy, his white blood cell count of 5750/μl was normal. After a course of treatment, his white blood cell count values were reduced at 1050/μl. His white blood cell count is raised to 6650/μl, while he starts to take the herbal extracts in the third embodiment of the present invention continuously following the end of chemotherapy. That also indicates the herbal extract has a significant role in preventing bone marrows suppression and bone marrow stem cells enhanced proliferation.

Furthermore, a pharmaceutical composition as defined in the specification refers to a mixture of the herbal composition with other chemical components, such as physiologically acceptable carriers and excipients. The purpose of a pharmacological composition is to facilitate administration of an extract or extracts of this invention to patients, e.g. the herbal extract said above is in a form of a oral dosage.

A physiologically acceptable carrier as defined in the specification refers to a carrier or diluent that does not cause significant irritation to an organism and does not abrogate the biological activity and properties of the administered composition.

An excipient as defined in the specification refers to an inert substance added to a pharmaceutical composition to further facilitate administration of the herbal composition. Examples, but not for limitation, of excipients include calcium carbonate, calcium phosphate, various sugars and types of starch, cellulose derivatives, gelatin, vegetable oils, and polyethylene glycols.

When the herbal composition is administered without combination with any other substances, the composition may be encased in a suitable capsule, such as a gelatin capsule. When administered in admixture with other excipients, adjuvants, binders, diluents, disintegrants, etc., the herbal composition may be compressed into a capsule, a tablet, a multiparticulate or a caplet in a conventional manner that is well-known in the art.

The above description should be considered as only the discussion of the preferred embodiments of the present invention. However, a person with an ordinary skill in the art may make various modifications to the present invention. Those modifications may be considered as still being fallen within the spirit and scope defined by the appended claims.

What is claimed is:

1. A herbal extract for inhibiting tumor growth, wherein the herbal extract is obtained by extracting a mixture of white castor and Formosanum elderberry by use of water and/or alcohol under a heating condition, wherein a weight ratio of white castor is 1 to 99% and a weight ratio of Formosanum elderberry is 99 to 1%.

2. A herbal extract as claimed in claim 1, wherein in the heating condition of extracting the mixture of white castor and Formosanum elderberry by use of water, the weight ratio of white castor to Formosanum elderberry is within the range of 1:1 to 1:2.

3. The herbal extract as claimed in claim 1, wherein in the heating condition of extracting the mixture of white castor and Formosanum elderberry by use of water, the weight ratio of white castor to Formosanum elderberry is within the range of 2:1 to 1:1.

4. The herbal extract as claimed in claim 1, wherein the heating condition of extracting the mixture of white castor and Formosanum elderberry by use of water is processed at a temperature above 70° C.

5. The herbal extract as claimed in claim 1, wherein the heating condition of extracting the mixture of white castor and Formosanum elderberry by use of water is processed at a temperature above 70° C. for over 60 minutes.

6. The herbal extract as claimed in claim 1, wherein a cancer is the one selected from a group consisting of leukemia, lymphoma, Hodgkin lymphomas, non-Hodgkin lymphomas, colorectal cancer, pancreatic cancer, breast cancer, ovarian cancer, prostate cancer, thyroid cancer, renal cell carcinoma, hepatoma, bile duct carcinoma, esophageal cancer, nasopharyngeal carcinoma, lung carcinoma, and any combination thereof.

7. The herbal extract as claimed in claim 6, wherein the herbal extract is used for reducing a size of a tumor.

8. The herbal extract as claimed in claim 1, wherein the herbal extract is in a form of a oral dosage.

9. The herbal extract as claimed in claim 8, wherein the oral dosage is in a form of a liquid, a powder, a tablet, a capsule or a multiparticulate dosage form.

10. A herbal extract for inhibiting tumor growth, wherein the herbal extract is obtained by extracting a mixture of white castor, Formosanum elderberry and pig bone by use of water and/or alcohol under a heating condition, wherein a weight ratio of white castor is 1 to 99%, a weight ratio of Formosanum elderberry is 99 to 1%, and a weight ratio of pig bone is 99 to 1%.
